(19) United States
(12) Patent Application Publication
(10) Pub. No.: US 2025/0003972 A1
(43) Pub. Date: Jan. 2, 2025

(54) A METHOD OF DETERMINING THE PRESENCE OF AN ANALYTE IN A SAMPLE

(71) Applicant: Valitacell Limited, Dublin (IE)

(72) Inventors: Ben Thompson, Sheffield (GB); Rory Vignoles, Dublin (IE); Hannah Byrne, Dublin (IE)

(21) Appl. No.: 18/690,064

(22) PCT Filed: Sep. 9, 2022
(86) PCT No.: PCT/EP2022/075183

§ 371 Date: Mar. 7, 2024

(30) Foreign Application Priority Data
Sep. 10, 2021 (EP) 21196085.1

(51) Int. Cl. G01N 33/58; G01N 21/64; G01N 33/68

(52) U.S. Cl. CPC G01N 33/582; G01N 21/6428; G01N 33/68; G01N 2021/6439

(57) ABSTRACT

A method for measuring the degree of aggregation of a protein or a polypeptide in a liquid sample, the method comprising the steps of combining an amount of a fluorophore molecule with the aggregated protein or polypeptide in the liquid sample; and measuring the fluorescence polarization value of the combination to determine the degree of aggregation: wherein the fluorophore molecule is a diazaoxatriangulenium of Formula (I) or a derivative thereof.

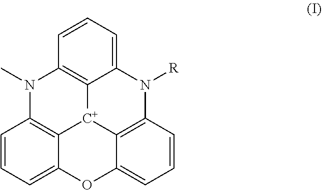

### FIELD OF THE INVENTION

The invention relates to determining the presence of an analyte in a sample using fluorescence polarisation (FP). Specifically, the invention relates to the detection of non-monomer formation in a sample using a fluorescent dye and FP.

### BACKGROUND TO THE INVENTION

Aggregation of therapeutic proteins during manufacture presents a significant risk to product success. This is the formation of high molecular weight species (HMWS), i.e., IgG molecules which are not monomeric.

Aggregates have been known to potentially affect the safety of a therapeutic and are thus considered a “critical quality attribute” (CQA) during manufacturing. Aggregation can potentially take place at many stages during the production process from cell culture to purification to drug formulation. Thus, the ability to measure aggregates rapidly and economically, is important.

The classic gold standard method to measure aggregates is via high performance liquid chromatography (HPLC) size exclusion chromatography (SEC). However, despite its accuracy, it is low through-put and requires highly trained personnel.

There exist several dye-based methods to measure level of aggregation, such as Sypro Orange, Thioflavin T and the commercially available Proteostat® (Enzo life sciences, Exeter, UK), which enable high throughput quantitation of aggregates via measurement of fluorescence. However, these suffer from poor accuracy when measuring low percentages of naturally occurring aggregates.

Work published by Sheun Oshinbolu et al. (Journal of Chemical Technology and Biotechnology. vol. 93 (3), pp. 909-917 (2018)) describes measurement of protein aggregation by using aggregation binding fluorophores. However, this study was performed using non-natural aggregates created by thermal denaturation. These dyes give poor performance using naturally occurring aggregates. Additionally, commercially available fluorophore based methods suffer from poor accuracy at low levels of aggregation with natural and non-natural aggregates.

It is an object of the present invention to overcome at least one of the above-mentioned problems.

### SUMMARY OF THE INVENTION

The Applicants have discovered that the derivatives of the fluorophore molecule diazaoxatriangulenium (DAOTA—Formulas (I) and (III)), such as N′-propyl, N-propyl derivative of diazaoxatriangulenium (N′PNP-DAOTA; Formula (II)), binds to non-monomer IgG in cell culture media, and that this binding can be measured with a high degree of sensitivity using fluorescence polarization (FP). Thus, this method can be used to quantitate the level of non-monomer (or other analytes) in cell culture supernatant.

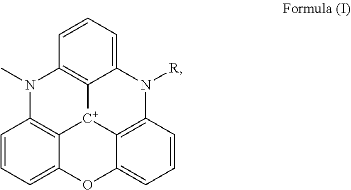

wherein R is independently selected from hydrogen, C₁-C₆ alkyl, C₂-C₆ alkenyl, C₂-C₆ alkynyl, phenyl, C₄-C₈ cycloalkyl, C₁-C₆ aminoalkyl, C₁-C₆ alkylenesulphonate, C₁-C₁₈ alkyl, C₁-C₂₂ alkyl, methylenesulfonate, ethylenesulfonate, C₁-C₆ alkylsulphonyl, triflouromethyl, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, thiocarbonyl, C₁-C₆-alkylthio, heteroaryl, cycloalkyl, phenyl, hydroxyphenyl, aminophenyl, amino-C₁-C₆-alkyl, heterocyclyl, polyethylene glycol, carboxylic acid, alkyl halide, acrylamide, activated ester of a carboxylic acid, hydroxy, aldehyde, sulfonate, amine, antigen, anhydride, aniline, aryl halide, azide, aziridine, boronate, carbodiimide, diazoalkane, epoxide, glycol, haloacetamide, halotriazine, hydrazine, hydroxylamine, imido ester, isocyanate, isothiocyanate, ketone, maleimide, phosphoramidite, sulfonyl halide, thiol group, butyric acid, and butanoic acid.

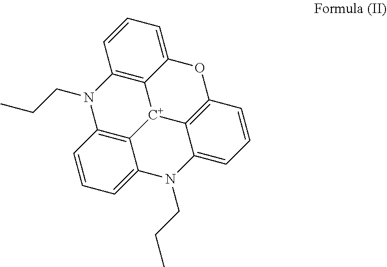 [SMILES: CCCn1c2cccc3oc4cccc5c4[c+](c32)c2c1cccc2n5CCC]

There is provided, according to the appended claims, a method of adding the fluorophore above to the antibody sample of interest and measuring the degree of aggregate binding using fluorescence polarization. The quantitative degree of aggregation of the sample can be determined by interpolating from a standard curve of known degree of aggregation.

There is provided a method for measuring the degree of aggregation of a protein or a polypeptide in a liquid sample, the method comprising the steps of:
adding an amount of a fluorophore molecule to the liquid sample comprising the aggregated protein or polypeptide;
measuring the fluorescence polarisation value of the liquid sample;
and comparing the measured fluorescence polarisation value of the liquid sample against a reference fluorescent polarisation value to determine the degree of aggregation of the protein or the polypeptide in the liquid sample;
wherein the fluorophore molecule is a diazaoxatriangulenium of Formula (I)

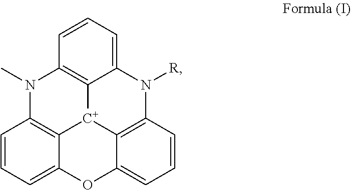

or a derivative thereof; and
wherein R is independently selected from hydrogen, C₁-C₆ alkyl, C₂-C₆ alkenyl, C₂-C₆ alkynyl, phenyl, C₄-C₈ cycloalkyl, C₁-C₆ aminoalkyl, C₁-C₆ alkylenesulphonate, C₁-C₁₈ alkyl, C₁-C₂₂ alkyl, methylenesulfonate, ethylenesulfonate, C₁-C₆ alkylsulphonyl, triflouromethyl, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, thiocarbonyl, C₁-C₆-alkylthio, heteroaryl, cycloalkyl, phenyl, hydroxyphenyl, aminophenyl, amino-C₁-C₆-alkyl, heterocyclyl, polyethylene glycol, carboxylic acid, alkyl halide, acrylamide, activated ester of a carboxylic acid, hydroxy, aldehyde, sulfonate, amine, antigen, anhydride, aniline, aryl halide, azide, aziridine, boronate, carbodiimide, diazoalkane, epoxide, glycol, haloacetamide, halotriazine, hydrazine, hydroxylamine, imido ester, isocyanate, isothiocyanate, ketone, maleimide, phosphoramidite, sulfonyl halide, thiol group, butyric acid, and butanoic acid.

In one aspect, the derivative is a diazaoxatriangulenium of:

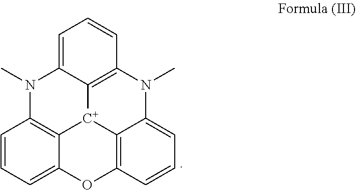 [SMILES: Cn1c2cccc3oc4cccc5c4[c+](c32)c2c1cccc2n5C]

In one aspect, the fluorophore molecule is selected from an N-propyl, N′-propyl derivative of diazaoxatriangulenium. Preferably, the fluorophore molecule is an N-propyl, N′-propyl derivative of diazaoxatriangulenium, as shown in Formula (II)

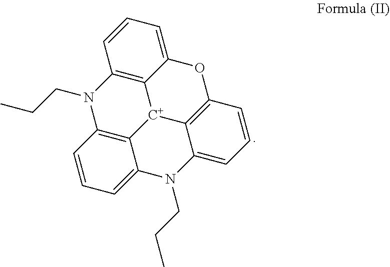 [SMILES: CCCn1c2cccc3oc4cccc5c4[c+](c32)c2c1cccc2n5CCC]

In one aspect, the protein or polypeptide is selected from an antibody, antibody fragments, enzymes, amyloids. Preferably, the antibody is selected from IgG, IgM, IgE, IgD and IgA.

In one aspect, there is provided a method of determining the presence of an aggregated analyte in a sample, the method comprising:
contacting the sample with a compound of Formula (I)

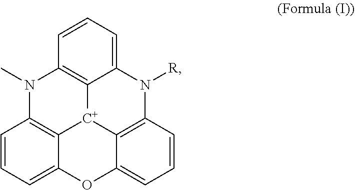

or a derivative thereof,
wherein R is independently selected from hydrogen, C₁-C₆ alkyl, C₂-C₆ alkenyl, C₂-C₆ alkynyl, phenyl, C₄-C₈ cycloalkyl, C₁-C₆ aminoalkyl, C₁-C₆ alkylenesulphonate, C₁-C₁₈ alkyl, C₁-C₂₂ alkyl, methylenesulfonate, ethylenesulfonate, C₁-C₆ alkylsulphonyl, triflouromethyl, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, thiocarbonyl, C₁-C₆-alkylthio, heteroaryl, cycloalkyl, phenyl, hydroxyphenyl, aminophenyl, amino-C₁-C₆-alkyl, heterocyclyl, polyethylene glycol, carboxylic acid, alkyl halide, acrylamide, activated ester of a carboxylic acid, hydroxy, aldehyde, sulfonate, amine, antigen, anhydride, aniline, aryl halide, azide, aziridine, boronate, carbodiimide, diazoalkane, epoxide, glycol, haloacetamide, halotriazine, hydrazine, hydroxylamine, imido ester, isocyanate, isothiocyanate, ketone, maleimide, phosphoramidite, sulfonyl halide, thiol group, butyric acid, and butanoic acid;
illuminating the sample;
measuring the fluorescence polarisation of the illuminated sample; and
comparing the fluorescence polarisation value of the illuminated sample against a reference fluorescence polarisation value to determine the degree of aggregation of the analyte in the sample.

In one aspect, the derivative is a diazaoxatriangulenium of Formula (III)

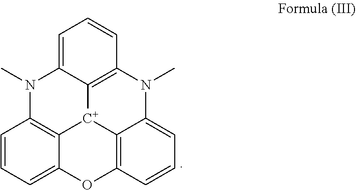 [SMILES: Cn1c2cccc3oc4cccc5c4[c+](c32)c2c1cccc2n5C]

In one aspect, the compound is an N-propyl, N′-propyl derivative of diazaoxatriangulenium. Preferably, the compound is an N-propyl, N′-propyl derivative of diazaoxatriangulenium, as shown in Formula (II)

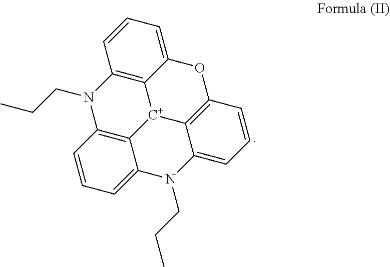 [SMILES: CCCn1c2cccc3oc4cccc5c4[c+](c32)c2c1cccc2n5CCC]

In one aspect, the analyte is selected from an antibody, an antibody fragment, an enzyme, and an amyloid.

In one aspect, the derivatives of Formula (I) may be conjugated to a carrier molecule, the carrier molecule preferably being selected from the group comprising an amino acid, a peptide, a protein, a polysaccharide, a nucleoside, a nucleotide, an oligonucleotide, a nucleic acid polymer, a drug, a hormone, a lipid, a lipid assembly, a synthetic polymer, a polymeric microparticle, a biological cell, or a virus. Other carrier molecules may also be suitable.

In one aspect, the derivatives of Formula (I) may be conjugated to a solid support by well-established methods known to the skilled person. Useful solid supports include solid and semisolid matrixes, such as sol-gels, aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

Conjugates of carrier molecules, e.g., drugs, peptides, toxins, nucleotides, phospholipids, proteins and other organic molecules including the derivatives of Formula (I) of the present invention, are generally prepared by means well recognized in the art (Haug-land, MOLECULAR PROBES HANDBOOK, supra, (2002)). Preferably, conjugation forming a covalent bond consists of simply mixing the reactive derivatives of the present invention in a suitable solvent in which both the reactive derivative and the substance to be conjugated are soluble. The reaction preferably proceeds spontaneously without added reagents at room temperature or below. For those reactive derivatives that are photoactivated, conjugation is facilitated by illumination of the reaction mixture to activate the reactive derivative. Chemical modification of water-insoluble substances, so that a desired derivative-conjugate may be prepared, is preferably performed in an aprotic solvent such as dimethylformamide, dimethylsulfoxide, acetone, ethyl acetate, toluene, or chloroform. Similar modification of water-soluble materials is readily accomplished through the use of the instant reactive derivatives to make them more readily soluble in organic solvents.

### Definitions

In the specification, the term “protein” and “polypeptide” should be understood to mean a large biomolecule and macromolecule that are comprised of one or more long chains of amino acid residues. Proteins differ from one another primarily in their sequence of amino acids. A linear chain of amino acid residues is called a polypeptide. A protein contains at least one long polypeptide. Short polypeptides, containing less than 20-30 residues, are rarely considered to be proteins and are commonly called peptides, or sometimes oligopeptides. The individual amino acid residues are bonded together by peptide bonds and adjacent amino acid residues. Examples of proteins and/or polypeptides include antibodies, antibody fragments, enzymes, Fc fusion proteins, anticoagulants (such as coumarins (vitamin K antagonist such as warfarin), heparin and derivative substances (unfractionated heparin (UFH), low molecular weight heparin (LMWH), and ultra-low-molecular weight heparin (ULMWH)), synthetic pentasaccharide inhibitors of factor Xa (fondaparinux, idraparinux, idrabiotaparinux), directly acting oral anticoagulants (DOACs; such as dabigatran, rivaroxaban, apixaban, edoxaban and betrixaban), antithrombins), direct factor Xa inhibitors (such as rivaroxaban, apixaban and edoxaban)), blood factors (I, II, III, IV, V, VI, VII, VIII, IX, X, XI, XII, XIII), bone morphogenetic proteins, engineered protein scaffolds, growth factors, hormones, interferons, interleukins, thrombolytics, amyloids (such as amyloid beta), tau, alpha-synuclein, TAR DNA binding protein 43, C9orf72-related proteins, islet amyloid polypeptide and transthyretin.

In the specification, the term “Fc fusion protein” (also known as Fc chimeric fusion proteins, Fc-Ig's, Ig-based Chimeric Fusion proteins and Fc-tag proteins) should be understood to mean the Fc domain of IgG genetically linked to a peptide or protein of interest.

Proteins and polypeptides (including variants and fragments thereof) for use in the invention may be generated wholly or partly by chemical synthesis or by expression from nucleic acid. The proteins and peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2^{nd} edition, Pierce Chemical Company, Rockford, Illinois (1984), in M. Bodanzsky and A. Bodanzsky, The Practice of Peptide Synthesis, Springer Verlag, New York (1984).

In the specification, the term “variant” should be understood to mean a protein or peptide obtained by introducing one or more substitution, addition and/or deletion into a sequence of a wild type protein or peptide. The term “variant” is also intended to include mimics (i.e., peptide mimics) and chemical derivatives of generic antibody binding protein, i.e. where one or more residues of the generic antibody binding protein is chemically derivatized by reaction of a functional side group. Also included within the term variant are generic antibody binding proteins are in which naturally occurring amino acid residues are replaced with amino acid analogues. Examples of generic antibody binding protein variants are described in Dinon et. al (J. Mol. Recognit. 2011 November-December; 24(6)).

In the specification, the term “fluorescent compound” or “fluorophore molecule” should be understood to mean a compound that when exposed to light of the proper wavelength, its presence can then be detected due to fluorescence. A detectable label can be a fluorescent dye molecule, or a fluorophore molecule.

In this specification, the term “antibody” should be understood to mean an immunoglobulin, for example an IgG, IgA, IgE or IgM immunoglobulin in a monoclonal or polyclonal form, or a fragment thereof, in a humanised or non-humanised. In one aspect, the antibody is an IgG molecule, preferably a monoclonal IgG molecule. In one aspect, the antibody is a human antibody.

In the specification, the term “amyloid” should be understood to mean an aggregate of proteins characterised by an extracellular, proteinaceous fibrillar deposit exhibiting β-sheet secondary structure and an ability to be stained by particular dyes, such as Congo red, where it is identifiable by an apple-green birefringence under polarized light). Amyloids are essentially any polypeptide that polymerizes to form a cross-β structure, in vivo or in vitro, inside or outside cells.

In the specification, the term “enzyme” should be understood to mean proteins that act as biological catalysts.

The term “fluorescence polarisation” should be understood to mean exciting a sample with plane polarised light at a wavelength corresponding to an excitation wavelength of a fluorescent dye, and detecting light intensity emitted by the fluorescent dye at an appropriate emission wavelength both in two planes, one of which is parallel to the plane of the excitation plane and one of which is perpendicular to the plane of the excitation light. The excitation plane is vertical or horizontal and the emitted light is detected in vertical and horizontal planes. The degree to which the emission intensity moves from the excitation plane (i.e., vertical) to a perpendicular plane (i.e., horizontal)—i.e., the change in polarisation between excitation and emission light—is a function of the degree of rotation of the fluorescent dye.

In the specification, the term “trianguleniums” should be understood to mean a dye that constitutes a family of versatile chromophores with impressive photo-absorption and emission properties. Typical members of tranguleniums are the aza/oxa-triangulenium dyes Azadioxatriangulenium (ADOTA⁺) and diazaoxatriangulenium (DAOTA⁺) (see Formula (III) below), which have remarkable low rates of non-radiative deactivation and low susceptibility to quenching by oxygen. These organic dyes have fluorescence peaks in the 550 nm-600 nm range, can display unusually long fluorescence lifetimes, close to 20 ns, and have high quantum yields. The aza/oxa-triangulenium dyes are highly stabilized carbenium ions, with a rigid and planar heterocyclic framework.

In the specification, the term “Alkyl” refers to monovalent saturated aliphatic hydrocarbyl groups. The term “C₁-C₆-alkyl” and “C₁-C₁₂-alkyl”, unless otherwise indicated, denotes an alkyl group with 1 to 6 or 1 to 12 carbon atoms, respectively. Suitable alkyl groups include straight or branched C₁-C₆-alkyl, which, unless otherwise indicated, denotes an alkyl group with 1 to 6 carbon atoms. Such suitable C₁-C₆-alkyl groups include, for example, methyl, ethyl, propyl, e.g., n-propyl and isopropyl, butyl, e.g., n-butyl, iso-butyl, sec-butyl and tert-butyl, pentyl, e.g., n-pentyl, and hexyl (e.g., n-hexyl). Suitable alkyl groups include linear C₁-C₁₂-alkyl, which, unless otherwise indicated, refers to straight alkyl chains of 1, 2, 3, 4, 5, 6, 7, 8, 9, 10, 11, or 12 carbon atoms.

In the specification, the term “Alkenyl” refers to aliphatic hydrocarbyl groups having at least one double bond. The term “C₂-C₆-alkenyl”, unless otherwise indicated, may be interpreted similarly to the term “alkyl”. Suitable alkenyl groups include, for example, ethenyl, propenyl, 1-butenyl, and 2-butenyl.

In the specification, the term “Alkynyl” refers to aliphatic hydrocarbyl groups having at least one double bond. The term “C₂-C₆-alkynyl”, unless otherwise indicated, may be interpreted similarly to the term “alkyl”. Alkenyl groups contain at least 1 triple bond. The term “halogen”, unless otherwise indicated, denotes fluorine (F), chlorine (Cl), bromine (Br) and iodine (I), preferably F, CI or Br. The compounds of Formula (I) and compounds of Formula (III) may be substituted with one, two, three, four, five, six or even more halogens, preferable CI or Br, more preferable Cl.

In the specification, the term “C₁-C₆-alkoxy” refers to the group —O—C₁-C₆-alkyl wherein C1-C₆-alkyl is defined herein. Alkoxy includes, by way of example, methoxy, ethoxy, n-propoxy, isopropoxy, n-butoxy, f-butoxy, sec-butoxy, and n-pentoxy.

In the specification, the term “C₁-C₁₂ alkanoic acid” refers to the group of C₁-C₁₂ alkyl COOH.

In the specification, the term “Acyl” refers to the groups H—C(O)—, alkyl-C(O)—, alkenyl-C(O)—, alkynyl-C(O)—, cycloalkyl-C(O)—, cycloalkenyl-C(O)—, aryl-C(O)—, heteroaryl-C(O)—, and heterocyclic-C(O)—, wherein alkyl, alkenyl, alkynyl, cycloalkyl, cycloalkenyl, aryl, heteroaryl, and heterocyclic are as otherwise defined herein. Acyl includes the “acetyl” group CH₃ C(O)—.

In the specification, the term “Acylamino” refers to the groups-NRC(0)alkyl, —NRC(O)cycloalkyl, —NRC(O)cycloalkenyl, —NRC(O)alkenyl, —NRC(O)alkynyl, —NRC(O)aryl, NRC(O)heteroaryl, and —NRC(O)heterocyclic, wherein R is hydrogen or alkyl and wherein alkyl, alkenyl, alkynyl, cycloalkyl, cycloalkenyl, aryl, heteroaryl, and heterocyclic are as otherwise defined herein,

In the specification, the term “Acyloxy” refers to the groups alkyl-C(O)O—, alkenyl-C(O)O—, alkynyl-C(O)O—, aryl-C(O)O—, cycloalkyl-C(O)O—, cycloalkenyl-C(O)O—, heteroaryl-C(O)O—, and heterocyclic-C(O)O—, wherein alkyl, alkenyl, alkynyl, cycloalkyl, cycloalkenyl, aryl, heteroaryl, and heterocyclic are as otherwise defined herein.

In the specification, the term “Aryl” refers to a monovalent aromatic carbocyclic group of from 5 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

In the specification, the term “Carboxyl” or “carboxy” refers to —COOH.

In the specification, the term “Carboxyl ester” or “carboxy ester” refers to the groups —C(O)O-alkyl, —C(O)O-alkenyl, —C(O)O-alkynyl, —C(O)O-aryl, —C(O)O-cycloalkyl, —C(O)O-cycloalkenyl, —C(O)O-heteroaryl, and —C(O)O-heterocyclic, wherein alkyl, alkenyl, alkynyl, cycloalkyl, cycloalkenyl, aryl, heteroaryl, and heterocyclic are as defined herein.

In the specification, the term “(Carboxyl ester)amino” refers to the group —NR—C(O)O-alkyl, —NR—C(O)O-alkenyl, —NR—C(O)O-alkynyl, —NR—C(O)O-aryl, —NR—C(O)O-cycloalkyl, —NR—C(O)O-cycloalkenyl, —NR—C(O)O-heteroaryl, and —NR—C(O)O-heterocyclic, wherein alkyl, alkenyl, alkynyl, cycloalkyl, cycloalkenyl, aryl, heteroaryl, and heterocyclic are as defined herein.

In the specification, the term “(Carboxyl ester)oxy” refers to the group —O—C(O)O-alkyl, s-O—C(O)O-alkenyl, —O—C(O)O-alkynyl, —O—C(O)O-aryl, —O—C(O)O-cycloalkyl, —O—C(O)O-cycloalkenyl, —O—C(O)O-heteroaryl, and —O—C(O)O-heterocyclic, wherein alkyl, alkenyl, alkynyl, cycloalkyl, cycloalkenyl, aryl, heteroaryl, and heterocyclic are as defined herein. “Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

In the specification, the term “Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

In the specification, the term “Heteroaryl” refers to an aromatic group of from 5 to 10 carbon atoms and 1 to 4 heteroatoms selected from the group consisting of oxygen, nitrogen and sulphur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. Preferred heteroaryls include pyridinyl, pyrrolyl, indolyl, thiophenyl, and furanyl.

In the specification, the term “Heterocycle” or “heterocyclic” or “heterocycloalkyl” or “heterocyclyl” refers to a saturated or unsaturated group having a single ring or multiple condensed rings, including fused bridged systems, from 1 to 10 carbon atoms and from 1 to 4 hetero atoms selected from the group consisting of nitrogen, sulphur or oxygen within the ring wherein, in fused ring systems, one or more the rings can be cycloalkyl, aryl or heteroaryl provided that the point of attachment is through the non-aromatic ring. In one embodiment, the nitrogen and/or sulphur atom(s) of the heterocyclic group are optionally oxidized to provide for the N-oxide, sulfinyl, sulfonyl moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

In the specification, the term “5- or 6-membered heterocyclyl containing at least one nitrogen or sulphur atom” include, but are not limited to, benzoefuran, indole, pyrrolidine, pyrrole, thiolane, thiophene, imidazolidine, pyrazolidine, imidazole, pyrazole, oxazolidine, isoxazolidine, oxazole, isoxazole, thiazolidine, isothiazolidine, thiazole, isothiazole, dithiolane, triazoles, furazan, oxadiazole, thiadiazole, dithiazole, tetrazole, piperidine, pyridine, thiane, thiopyran, piperazine, diazines, morpholine, oxazine, thiomorpholine, thiazine, dithiane, dithiine, triazine, or tetrazine.

In the specification, the term “Sulphonate” refers to the group —S(O)₃—; while the term “Sulfonyl” refers to the divalent group —S(O)₂—.

In the specification, the term “Alkylsulfonyl” refers to the group —S(O)₂-alkyl wherein alkyl is as defined herein. Preferably, the alkyl group is a small group having less than 6 carbon atoms, preferably the alkyl group is methyl or ethyl.

In the specification, the term “Sulfonyloxy” refers to the group —OSO₂-alkyl, —OSO₂-alkenyl —OSO₂-cycloalkyl, —OSO₂-cycloalkenyl, —OSO₂-aryl, —OSO₂-heteroaryl, and —OSO₂-heterocyclic, wherein alkyl, alkenyl, alkynyl, cycloalkyl, cycloalkenyl, aryl, heteroaryl, and heterocyclic are as defined herein.

In the specification, the term “Thioacyl” refers to the groups H—C(S)—, alkyl-C(S)—, alkenyl-C(S)—, alkynyl-C(S)—, cycloalkyl-C(S)—, cycloalkenyl-C(S)—, aryl-C(S)—, heteroaryl-C(S)—, and heterocyclic-C(S)—, wherein alkyl, alkenyl, alkynyl, cycloalkyl, cycloalkenyl, aryl, heteroaryl, and heterocyclic are as defined herein.

In the specification, the term “Thiol” refers to the group —SH.

In the specification, the term “Thiocarbonyl” refers to the divalent group —C(S)— which is equivalent to —C(═S)—.

In the specification, the term “Alkylthio” refers to the group-S-alkyl wherein alkyl is as defined herein.

In the specification, the term “conjugated substance” as used herein refers to a biological or a non-biological component that is or becomes attached to a compound of Formula (I) of the present invention.

In the specification, the term “reference fluorescent polarisation value” when used in the present invention refers to a list, standard curve, or database of known fluorescent polarisation values that were measured when the fluorophore molecule or compound used in the claimed invention was mixed with and bound to a known concentration of aggregated protein, aggregated polypeptides, and aggregated analytes in a liquid sample. This standard curve or database of known fluorescent polarisation values is used as a reference point for the method of the claimed invention to determine the concentration of aggregated protein, polypeptide or analyte in a liquid sample being tested.

In the specification, the term “database” insofar as it relates to calibrating for measuring a degree of aggregation in a sample, should be understood to mean to a standard curve of known fluorescence polarisation values for known % aggregation in a sample. The use of a standard curve (a calibration curve, or a reference curve) is common and standard practice when using assays to determine the concentration or % aggregation of a protein of interest in an unknown sample. The term “database” should thus also be referred to as a standard curve or a reference curve or calibration curve of known fluorescence polarisation values for known % aggregation in a sample. The database used in the subject application is determined as follows: for generation of a standard curve or a calibration curve, a range of the protein of interest with a known degree of aggregation (e.g., the degree of aggregation has been pre-quantified using size exclusion high-performance liquid chromatography (HPLC-SEC)) is prepared and the assay is performed, with the results plotted as per FIG. 1. The mP values for each degree of aggregation is measured, as demonstrated in FIG. 1. This is the practice of producing a “standard curve” or “calibration curve”, a method well known to those skilled in the art of performing assays. Next, when a sample of unknown aggregation is measured, its mP values can be interpolated into a proportion of aggregation, for example, by using a standard curve or a calibration curve, such as that presented in FIG. 1. The specific values for the standard curve (e.g., proportion of aggregation for the protein of interest and its corresponding mP values) can be stored (either computationally or manually) and re-used as a reference, or alternatively, the standard curve can be generated again alongside the unknown samples within the same experiment.

### BRIEF DESCRIPTION OF THE DRAWINGS

The invention will be more clearly understood from the following description of an embodiment thereof, given by way of example only, with reference to the accompanying drawings, in which: —

FIG. 1 illustrates that high molecular weight species (HMWS) of IgG (non-monomer) was spiked into monomer IgG in various proportions in 120 ul total of culture media (CD-CHO, ThermoFisher, UK). 0.1 ug of an N′-propyl, N-propyl derivative of diazaoxatriangulenium (N′PNP-DAOTA) was added, and the polarization signal was measured after 5 minutes incubation in the dark.

FIG. 2 illustrates that high molecular weight species (HMWS) of IgG (non-monomer) was spiked into monomer IgG in various proportions in culture media. This highlights the relationship between aggregation and assay signal for the competitor proteostat enzo dye.

FIG. 3 illustrates a comparison of different derivatives of diazaoxatriangulenium (DAOTA) and their binding characteristics as shown by their polarisation signals.

FIG. 4 illustrates a comparison of different derivatives of azadioxatriangulenium (ADOTA) and aggregation with monomer IgG, as shown by their polarisation signals.

FIG. 5 illustrates a performance study using commercially available aggregation standards where (A) is DAOTA predicted % aggregation versus known % aggregation and (B) is the Enzo Proteostat® assay predicted % aggregation versus known % aggregation. As is clearly set out in FIG. 5 is that (A) DAOTA used in the claimed invention outperforms (B) when the Enzo Proteostat® assay is for measuring non-native aggregates in solution, with an R2 of 0.99 versus 0.93 respectively.

FIG. 6 is a pair of graphs illustrating (A) DAOTA predicted % of natural non-monomer versus known % non-monomer (using method of claimed invention) and (B) use of Enzo Proteostat® assay for predicted % non-monomer versus known % non-monomer. As is quite evident, DAOTA can measure native aggregates in solution (see (A)). FIG. 6(A) demonstrates the performance of the DAOTA for quantifying % dimer from monomer samples spiked with varying % dimer (HPLC-SEC characterized and separated). The correlation between DAOTA and the known % dimer is excellent with an R2=0.99. In contrast, the Enzo Proteostat® assay (FIG. 6(B)) cannot differentiate dimer from monomer or any native aggregate.

### DETAILED DESCRIPTION OF THE DRAWINGS

### Materials and Methods

N′PNP-DAOTA (see Formula (II)) was purchased from Ku-Dyes (Copenhagen, Denmark).

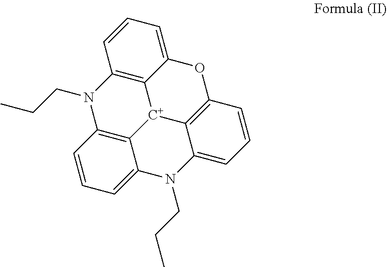 [SMILES: CCCn1c2cccc3oc4cccc5c4[c+](c32)c2c1cccc2n5CCC]

Derivatives of DAOTA were generated by adding different R groups to the structure of Formula (I).

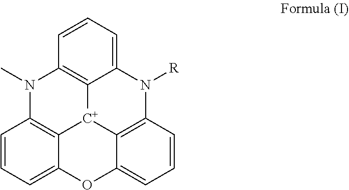

Derivatives of ADOTA were generated by adding different R groups to the structure of Formula (IV).

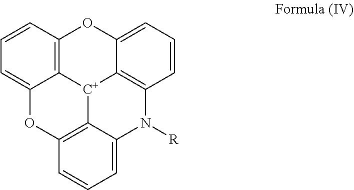

Four different R groups were used in this study: polyethylene glycol (PEG), butyric acid, N′PNP and C18 (18 carbon chain).

Non-monomer IgG was sourced internally. This was obtained by size exclusion chromatography from Chinese Hamster Ovary (CHO) cell supernatant (this is a naturally occurring non-monomer IgG).

FP readings were made using an BMG Pherastar plate reader (BMG, Berlin, Germany) in black half area NBS plates in a total of 120 ul (Corning, New York, USA).

The user instructions for the Enzo Proteostat® assay were followed and the fluorescence was measured. Briefly, 60 ul IgG sample, and 38 ul media, and 2 ul probe solution were added to each well. This was incubated at room temperature for 15 minutes prior to reading fluorescence.

For DAOTA FP assays, high molecular weight species (HMWS) of IgG (non-monomer) was spiked into monomer IgG in various proportions in 120 ul total of culture media (CD-CHO, ThermoFisher, UK) at a constant at a concentration of 200 mg/L. 0.1 ug of an N′-propyl, N-propyl derivative of diazaoxatriangulenium (N′PNP-DAOTA) was added, and the polarization signal was measured after 5 minutes incubation in the dark.

### Results

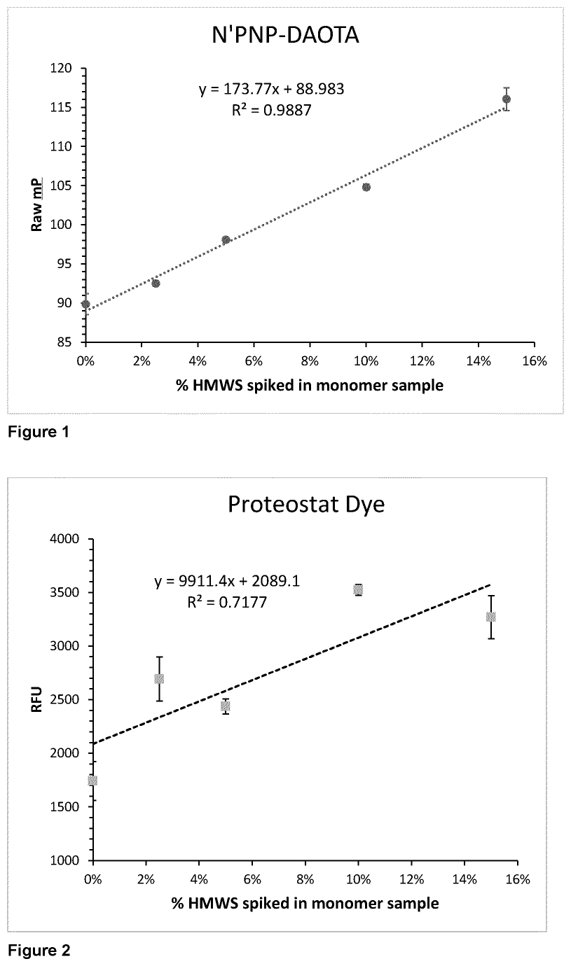

It is clear from FIG. 1 that N′PNP-DAOTA can be used to accurately measure the degree of non-monomer IgG in cell culture media. FIG. 2 is a comparison with the Enzo Proteostat® assay, which is similar in method but relies on a fluorescence signal rather than a fluorescence polarization signal.

Interestingly, when a very similar triangulenium molecule, N-butanoic acid aza-di-oxa-triangulenium (N-Phenyl ADOTA), was used instead of N′PNP-DAOTA, N-Phenyl-ADOTA did not bind to IgG aggregates despite its similar structure.

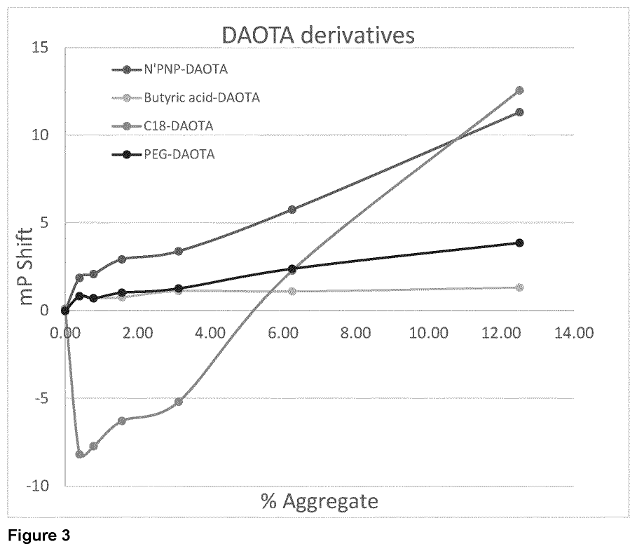

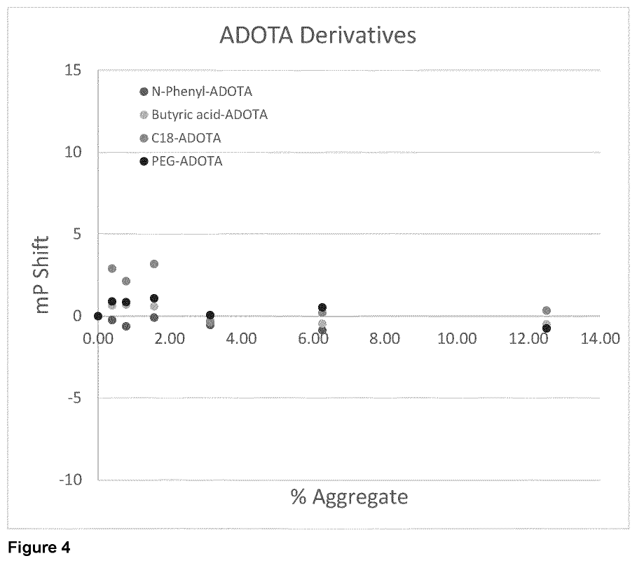

To determine what effect a side chain group attached to the fluorophore has, various side chains were added to the fluorophore of Formula (I) and Formula (IV). The concerns were whether the effects were limited to the compound with the N′PNP side groups The aggregation binding effects of the side chain groups on the DAOTA molecule were compared to the effects of the side chain groups on the ADOTA molecule (see Formula (IV). FIG. 3 and FIG. 4 show the aggregation binding is very specific to the DAOTA molecule. All side chains permit IgG aggregate binding with DAOTA albeit with varying shifts. Conversely, regardless of side chain modification, no IgG aggregate binding was observed with the ADOTA derivatives as there is no clear relationship between the percentage non monomer and the FP (mP) signal.

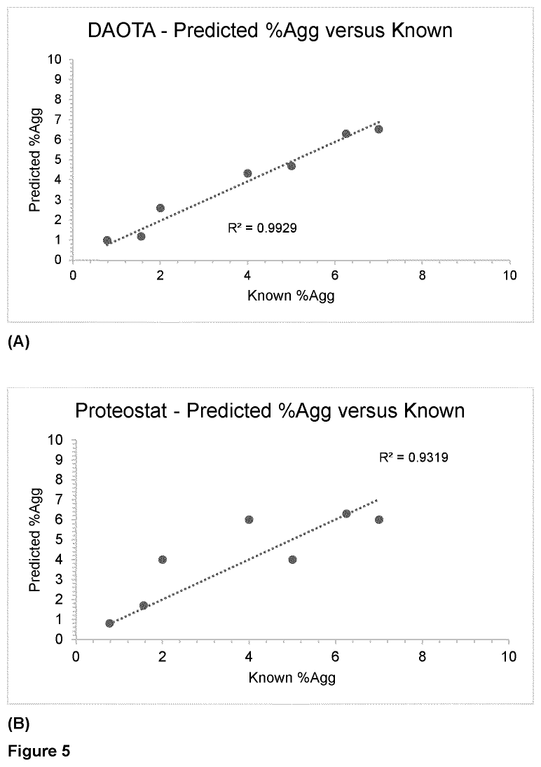

Performance studies carried out using commercially available aggregation standards, demonstrate that DAOTA (FIG. 5(A)) outperforms the Enzo Proteostat® assay, when following their protocol as advised (FIG. 5(B)) for measuring non-native aggregates in solution with an R² of 0.99 versus 0.93 respectively.

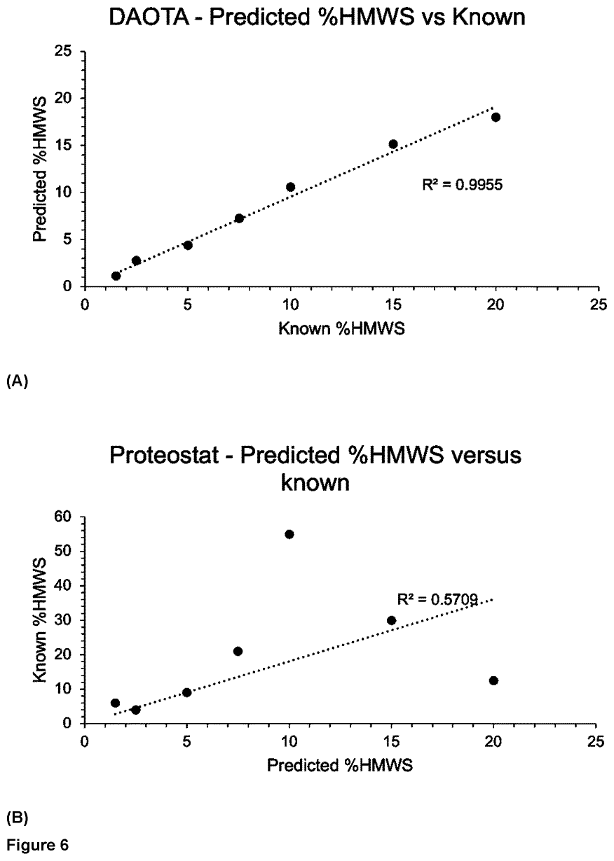

The DAOTA molecule can also measure native aggregates in solution, as shown in FIG. 6. FIG. 6(A) demonstrates the performance of the DAOTA molecule for quantifying % dimer from monomer samples spiked with varying % dimer (HPLC-SEC characterized and separated). The correlation between the DAOTA molecule and the known % dimer is far superior, with an R²=0.99. In contrast, the Enzo Proteostat® assay (FIG. 6(B)) cannot differentiate dimer from monomer, or any native aggregate given the poor R² value of 0.57.

### Discussion

The Applicants have shown that N′PNP-DAOTA and various variants of DAOTA binds to non-monomer IgG in cell culture media, and that this binding can be measured with a high degree of sensitivity using FP. Thus, this method can be used to quantitate the level of non-monomer aggregates of proteins or polypeptides in cell culture supernatant.

When one compares the R² values between the results shown in FIG. 1 and FIG. 2, the R² value of 0.99 achieved using the method of the claimed invention is far superior to the R² value of 0.72 obtained using the Enzo Proteostat® assay, which is a gold standard assay currently on the market. This increase in low end accuracy is also apparent from FIG. 5. These results clearly illustrate an increased accuracy at low ranges of aggregation, and that this effect is highly specific to DAOTA and DAOTA derivatives. This is a surprising and unexpected result since no aggregate binding is observed using a very similar molecule, ADOTA, regardless of R group used.

The increase in accuracy of the claimed invention when compared to current gold standard fluorophore assay Enzo Proteostat® is even more profound when using naturally occurring IgG aggregates (see FIG. 6). The R² values of our invention vs the competitor are 0.996 and 0.571 respectively. The quantification of naturally occurring aggregates is clearly a truer representation of assay utility since aggregates measured with this assay will be naturally occurring, e.g., those from cell culture supernatants. Of note, Enzo Proteostat® is claimed to be far superior to Thioflavin T (as discussed in Oshinbolu et al) from their website (https://www.enzolifesciences.com/ENZ-51023/proteostat-protein-aggregation-assay/).

The specific fluorophore used in the claimed invention clearly excels in the differentiation of monomer from dimer (aggregates) above what one would expect, especially when compared to known fluorophore assay products.

In the specification the terms “comprise, comprises, comprised and comprising” or any variation thereof and the terms “include, includes, included and including” or any variation thereof are totally interchangeable, and they should all be afforded the widest possible interpretation and vice versa.

The invention is not limited to the embodiments hereinbefore described but may be varied in both construction and detail.

1. A method for measuring the degree of aggregation of a protein or a polypeptide in a liquid sample, the method comprising the steps of:
adding an amount of a fluorophore molecule to the liquid sample comprising the aggregated protein or polypeptide;
measuring the fluorescence polarisation value of the liquid sample;
and comparing the measured fluorescence polarisation value of the liquid sample against a reference fluorescent polarisation value to determine the degree of aggregation of the protein or the polypeptide in the liquid sample;
wherein the fluorophore molecule is a diazaoxatriangulenium of Formula (I) 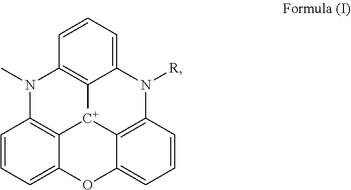
or a derivative thereof, wherein R is independently selected from hydrogen, C₁-C₆ alkyl, C₂-C₆ alkenyl, C₂-C₆ alkynyl, phenyl, C₄-C₈ cycloalkyl, C₁-C₆ aminoalkyl, C₁-C₆ alkylenesulphonate, C₁-C₁₈ alkyl, C₁-C₂₂ alkyl, methylenesulfonate, ethylenesulfonate, C₁-C₆ alkylsulphonyl, triflouromethyl, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, thiocarbonyl, C₁-C₆-alkylthio, heteroaryl, cycloalkyl, phenyl, hydroxyphenyl, aminophenyl, amino-C₁-C₆-alkyl, heterocyclyl, polyethylene glycol, carboxylic acid, alkyl halide, acrylamide, activated ester of a carboxylic acid, hydroxy, aldehyde, sulfonate, amine, antigen, anhydride, aniline, aryl halide, azide, aziridine, boronate, carbodiimide, diazoalkane, epoxide, glycol, haloacetamide, halotriazine, hydrazine, hydroxylamine, imido ester, isocyanate, isothiocyanate, ketone, maleimide, phosphoramidite, sulfonyl halide, thiol group, butyric acid, and butanoic acid.

2. The method according to claim 1, wherein the derivative is a diazaoxatriangulenium of

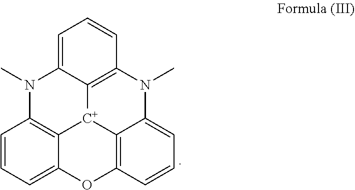 [SMILES: Cn1c2cccc3oc4cccc5c4[c+](c32)c2c1cccc2n5C]

3. The method according to claim 1 or claim 2, wherein the fluorophore molecule is selected from an N-propyl, N′-propyl derivative of diazaoxatriangulenium.

4. The method according to claim 3, wherein the fluorophore molecule is an N-propyl, N′-propyl derivative of diazaoxatriangulenium, as shown in Formula (II)

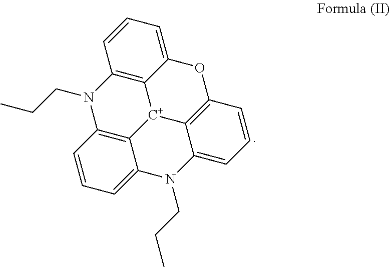 [SMILES: CCCn1c2cccc3oc4cccc5c4[c+](c32)c2c1cccc2n5CCC]

5. The method of any one of the preceding claims, wherein the protein or polypeptide is selected from an antibody, an antibody fragment, an enzyme, an amyloid, an Fc fusion protein, an anticoagulant, a blood factor, a bone morphogenetic protein, an engineered protein scaffold, a growth factor, a hormone, an interferon, an interleukin, a thrombolytic, tau, alpha-synuclein, TAR DNA binding protein 43, a C9orf72-related protein, islet amyloid polypeptide, and transthyretin.

6. The method according to claim 5, where in the antibody is selected from IgG, IgM, IgE, IgD and IgA.

7. A method of determining the presence of an aggregated analyte in a sample, the method comprising:
contacting the sample with a compound of Formula (I) 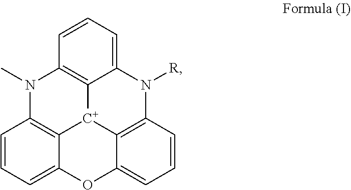
or a derivative thereof; wherein R is independently selected from hydrogen, C₁-C₆ alkyl, C₂-C₆ alkenyl, C₂-C₆ alkynyl, phenyl, C₄-C₈ cycloalkyl, C₁-C₆ aminoalkyl, C₁-C₆ alkylenesulphonate, C₁-C₁₈ alkyl, C₁-C₂₂ alkyl, methylenesulfonate, ethylenesulfonate, C₁-C₆ alkylsulphonyl, triflouromethyl, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, thiocarbonyl, C₁-C₆-alkylthio, heteroaryl, cycloalkyl, phenyl, hydroxyphenyl, aminophenyl, amino-C₁-C₆-alkyl, heterocyclyl, polyethylene glycol, carboxylic acid, alkyl halide, acrylamide, activated ester of a carboxylic acid, hydroxy, aldehyde, sulfonate, amine, antigen, anhydride, aniline, aryl halide, azide, aziridine, boronate, carbodiimide, diazoalkane, epoxide, glycol, haloacetamide, halotriazine, hydrazine, hydroxylamine, imido ester, isocyanate, isothiocyanate, ketone, maleimide, phosphoramidite, sulfonyl halide, thiol group, butyric acid, and butanoic acid;
illuminating the sample;
measuring the fluorescence polarisation of the illuminated sample; and
comparing the fluorescence polarisation value of the illuminated sample against a reference fluorescence polarisation value to determine the degree of aggregation of the analyte in the sample.

8. The method according to claim 7, wherein the derivative is a diazaoxatriangulenium of Formula (III)

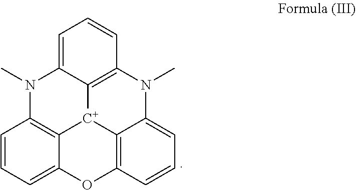 [SMILES: Cn1c2cccc3oc4cccc5c4[c+](c32)c2c1cccc2n5C]

9. The method according to claim 7 or claim 8, wherein the compound is an N-propyl, N′-propyl derivative of diazaoxatriangulenium.

10. The method according to claim 9, wherein the compound is an N-propyl, N′-propyl derivative of diazaoxatriangulenium, as shown in Formula (II)

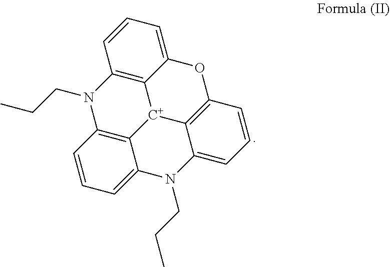 [SMILES: CCCn1c2cccc3oc4cccc5c4[c+](c32)c2c1cccc2n5CCC]

11. The method of any one of the preceding claims, wherein the analyte is selected from an antibody, an antibody fragment, an enzyme, an amyloid, an Fc fusion protein, an anticoagulant, a blood factor, a bone morphogenetic protein, an engineered protein scaffold, a growth factor, a hormone, an interferon, an interleukin, a thrombolytic, tau, alpha-synuclein, TAR DNA binding protein 43, a C9orf72-related protein, islet amyloid polypeptide, and transthyretin.
